(19) United States
(12) Patent Application Publication
(10) Pub. No.: US 2006/0167385 A1
(43) Pub. Date: Jul. 27, 2006

(54) Analysis of auscultatory sounds using voice recognition

(75) Inventor: Marie A. Guion, St. Paul, MN (US)

Correspondence Address: SHUMAKER & SIEFFERT, P. A., 8425 SEASONS PARKWAY, SUITE 105, ST. PAUL, MN, 55125, US

(73) Assignee: 3M Innovative Properties Company, St. Paul, MN (US)

(21) Appl. No.: 11/217,129

(22) Filed: Aug. 31, 2005

Related U.S. Application Data
(60) Provisional application No. 60/646,260, filed on Jan. 24, 2005.
(60) Provisional application No. 60/670,345, filed on Apr. 12, 2005.

(51) Int. Cl. A61B 7/00

(52) U.S. Cl. 600/586

(57) ABSTRACT

Techniques are described for analyzing auscultatory sounds to aid a medical professional in diagnosing physiological conditions of a patient. A data analysis system, for example, applies voice recognition and principle component analysis (e.g., singular value decomposition) to auscultatory sounds associated known physiological conditions to define a set of one or more disease regions within a multidimensional space. A diagnostic device, such as an electronic stethoscope or personal digital assistant, applies configuration data from the data analysis system to generate a set of one or more vectors within the multidimensional space representative of auscultatory sounds associated with a patient. The diagnostic device outputs a diagnostic message associated with a physiological condition of the patient based on the orientation of the vectors relative to the disease regions within the multidimensional space.

This application claims priority from U.S. Provisional Application Ser. No. 60/646,260, filed Jan. 24, 2005, and U.S. Provisional Application Ser. No. 60/670,345, filed Apr. 12, 2005, the entire contents of which are incorporated herein by reference.

### TECHNICAL FIELD

The invention relates generally to medical devices and, in particular, electronic devices for analysis of auscultatory sounds.

### BACKGROUND

Clinicians and other medical professionals have long relied on auscultatory sounds to aid in the detection and diagnosis of physiological conditions. For example, a clinician may utilize a stethoscope to monitor heart sounds to detect cardiac diseases. As other examples, a clinician may monitor sounds associated with the lungs or abdomen of a patient to detect respiratory or gastrointestinal conditions.

Automated devices have been developed that apply algorithms to electronically recorded auscultatory sounds. One example is an automated blood-pressure monitoring device. Other examples include analysis systems that attempt to automatically detect physiological conditions based on the analysis of auscultatory sounds. For example, artificial neural networks have been discussed as one possible mechanism for analyzing auscultatory sounds and providing an automated diagnosis or suggested diagnosis.

Using these conventional techniques, it is often difficult to provide an automated diagnosis of a specific physiological condition based on auscultatory sounds with any degree of accuracy. Moreover, it is often difficult to implement the conventional techniques in a manner that may be applied in real-time or pseudo real-time to aid the clinician.

### SUMMARY

In general, the invention relates to techniques for analyzing auscultatory sounds to aid a medical professional in diagnosing physiological conditions of a patient. The techniques may be applied, for example, to aid a medical profession in diagnosing a variety of cardiac conditions. Example cardiac conditions that may be automatically detected using the techniques described herein include aortic regurgitation and stenosis, tricuspid regurgitation and stenosis, pulmonary stenosis and regurgitation, mitrial regurgitation and stenosis, aortic aneurisms, carotid artery stenosis and other cardiac pathologies. The techniques may be applied to auscultatory sounds to detect other physiological conditions. For example the techniques may be applied to detect sounds recorded from a patient's lungs, abdomen or other areas to detect respiratory or gastrointestinal conditions.

In accordance with the techniques described herein, singular value decomposition (“SVD”) is applied to clinical data that includes digitized representations of auscultatory sounds associated with known physiological conditions. The clinical data may be formulated as a set of matrices, where each matrix stores the digital representations of auscultatory sounds associated with a different one of the physiological conditions. Application of SVD to the clinical data decomposes the matrices into a set of sub-matrices that define a set of “disease regions” within a multidimensional space.

One or more of the sub-matrices for each of the physiological conditions may then be used as configuration data within a diagnostic device. More specifically, the diagnostic device applies the configuration data to a digitized representation of auscultatory sounds associated with a patient to generate a set of one or more vectors within the multidimensional space. The diagnostic device determines whether the patient is experiencing a physiological condition, e.g., a cardiac pathology, based on the orientation of the vectors relative to the defined disease regions. In one embodiment, a method comprises applying voice recognition to auscultatory sounds associated with known physiological conditions to generate voice recognition coefficients; and mapping the coefficients to a set of one or more disease regions defined within a multidimensional space.

In another embodiment, a method comprises applying singular value decomposition (“SVD”) to digitized representations of auscultatory sounds associated with physiological conditions to map the auscultatory sounds to a set of one or more disease regions within a multidimensional space, and outputting configuration data for application by a diagnostic device based on the multidimensional mapping.

In another embodiment, a method comprises storing within a diagnostic device configuration data generated by the application of of voice recognition techniques and principle component analysis (PCA) to digitized representations of auscultatory sounds associated with known physiological conditions, wherein the configuration data maps the auscultatory sounds to a set of one or more disease regions within a multidimensional space. The method further comprises applying the configuration data to a digitized representation representative of auscultatory sounds associated with a patient to select one or more of the physiological conditions; and outputting a diagnostic message indicating the selected physiological conditions.

In another embodiment, a diagnostic device comprises a medium and a control unit. The medium stores data generated by the application of voice recognition to digitized representations of auscultatory sounds associated with known physiological conditions. The control unit applies the configuration data to a digitized representation representative of auscultatory sounds associated with a patient to select one of the physiological conditions. The control unit outputs a diagnostic message indicating the selected one of the physiological conditions.

In another embodiment, a data analysis system comprises an analysis module and a database. The analysis module applies voice recognition and principle component analysis (PCA) to digitized representations of auscultatory sounds associated with known physiological conditions to map the auscultatory sounds to a set of one or more disease regions within a multidimensional space. The database stores data generated by the analysis module.

In another embodiment, the invention is directed to a computer-readable medium containing instructions. The instructions cause a programmable processor to apply configuration data to a digitized representation representative of auscultatory sounds associated with a patient to select one of a set of physiological conditions, wherein the configuration maps the auscultatory sounds to a set of one or more disease regions within a multidimensional space using voice recognition and principle component analysis (PCA). The instructions further cause the programmable processor to output a diagnostic message indicating the selected one of the physiological conditions.

The techniques may offer one or more advantages. For example, the application of SVD may achieve more accurate automated diagnosis of the patient relative to conventional approaches. In addition, techniques allow configuration data to be pre-computed using the SVD, and then applied by a diagnostic device in real-time or pseudo real-time, i.e., by a clinician, to aid the clinician in rendering a diagnosis for the patient.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a block diagram illustrating an example system in which a diagnostic device analyzes auscultatory sounds in accordance with the techniques described herein to aid a clinician in rendering a diagnosis for a patient.

FIG. 2 is a block diagram of an exemplary embodiment of a portable digital assistant (PDA) operating as a diagnostic device in accordance with the techniques described herein.

FIG. 3 is a perspective diagram of an exemplary embodiment of an electronic stethoscope operating as a diagnostic device.

FIG. 4 is a flowchart that provides an overview of the techniques described herein.

FIG. 5 is a flowchart illustrating a parametric analysis stage in which singular value decomposition is applied to clinical data.

FIG. 6 is a flowchart that illustrates exemplary pre-processing of an auscultatory sound recording.

FIG. 7 is a graph that illustrates an example result of wavelet analysis and energy thresholding while pre-processing the auscultatory sound recording.

FIG. 8 illustrates an example data structure of an auscultatory sound recording.

FIG. 9 is a flowchart illustrating a real-time diagnostic stage in which a diagnostic device applies configuration data from the parametric analysis stage to provide a recommended diagnosis for a digitized representation of auscultatory sounds of a patient.

FIGS. 10A and 10B are graphs that illustrate exemplary results of the techniques by comparing aortic stenosis data to normal data.

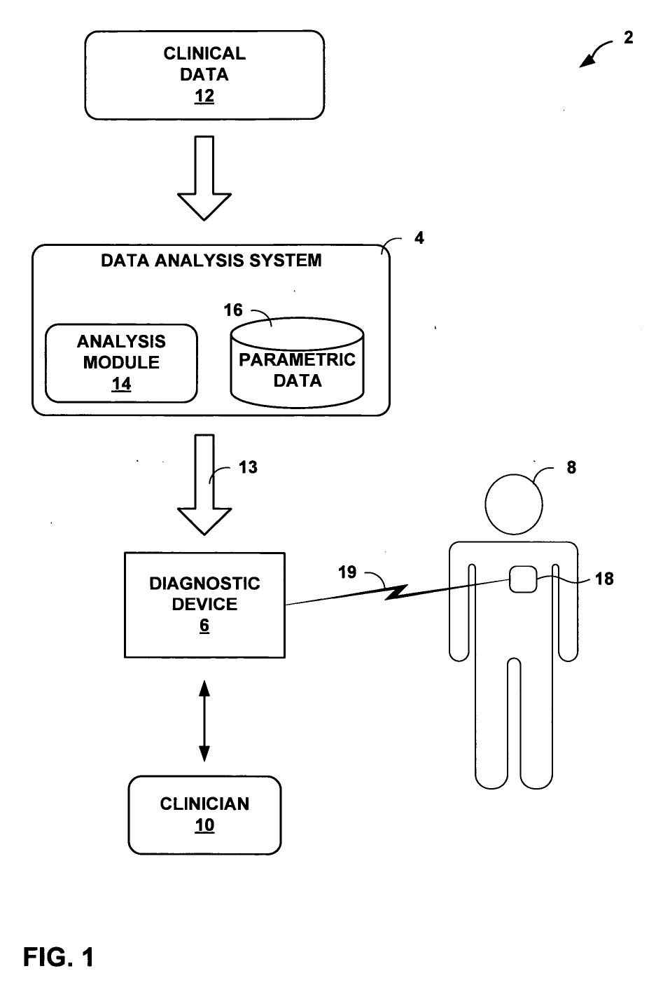

FIGS. 1A and 1B are graphs that illustrate exemplary results of the techniques by comparing tricuspid regurgitation data to normal data.

FIGS. 12A and 12B are graphs that illustrate exemplary results of the techniques by comparing aortic stenosis data to tricuspid regurgitation data.

FIG. 13 is a flowchart that illustrates another exemplary technique in which voice recognition techniques are used to pre-process the auscultatory sound recording prior to application of SVD.

FIGS. 14-17 are exemplary graphs that illustrate the use of voice recognition techniques and, in particular, mel-cepstrum coefficients for computing a disease within multi-dimensional space.

### DETAILED DESCRIPTION

FIG. 1 is a block diagram illustrating an example system 2 in which a diagnostic device 6 analyzes auscultatory sounds from patient 8 to aid clinician 10 in rendering a diagnosis. In general, diagnostic device 6 is programmed in accordance with configuration data 13 generated by data analysis system 4. Diagnostic device 6 utilizes the configuration data to analyze auscultatory sounds from patient 8, and outputs a diagnostic message based on the analysis to aid clinician 10 in diagnosing a physiological condition of the patient. Although described for exemplary purposes in reference to cardiac conditions, the techniques may be applied to auscultatory sounds recorded from other areas of the body of patient 8. For example, the techniques may be applied to auscultatory sounds recorded from the lungs or abdomen of patient 8 to detect respiratory or gastrointestinal conditions.

In generating configuration data 13 for application by diagnostic device 6, data analysis system 4 receives and processes clinical data 12 that comprises digitized representations of auscultatory sounds recorded from a set of patients having known physiological conditions. For example, the auscultatory sounds may be recorded from patients having one or more known cardiac pathologies. Example cardiac pathologies include aortic regurgitation and stenosis, tricuspid regurgitation and stenosis, pulmonary stenosis and regurgitation, mitrial regurgitation and stenosis, aortic aneurisms, carotid artery stenosis and other pathologies. In addition, clinical data 12 includes auscultatory sounds recorded from “normal” patients, i.e., patients having no cardiac pathologies. In one embodiment, clinical data 12 comprises recordings of heart sounds in raw, unfiltered format.

Analysis module 14 of data analysis system 4 analyzes the recorded auscultatory sounds of clinical data 12 in accordance with the techniques described herein to define a set of “disease regions” within a multi-dimensional energy space representative of the electronically recorded auscultatory sounds. Each disease region within the multidimensional space corresponds to characteristics of the sounds within a heart cycle that have been mathematically identified as indicative of the respective disease.

As described in further detail below, in one embodiment analysis module 14 applies singular value decomposition (“SVD”) to define the disease regions and their boundaries within the multidimensional space. Moreover, analysis module 14 applies SVD to maximize energy differences between the disease regions within the multidimensional space, and to define respective energy angles for each disease region that maximizes a normal distance between each of the disease regions. Data analysis system 4 may include one or more computers that provide an operating environment for execution of analysis module 14 and the application of SVD, which may be a computationally-intensive task. For example, data analysis system 4 may include one or more workstations or a mainframe computer that provide a mathematical modeling and numerical analysis environment.

Analysis module 14 stores the results of the analysis within parametric database 16 for application by diagnostic device 6. For example, parametric database 16 may include data for diagnostic device 6 that defines the multi-dimensional energy space and the energy regions for the disease regions with the space. In other words, the data may be used to identify the characteristics of the auscultatory sounds for a heart cycle that are indicative of normal cardiac activity and the defined cardiac pathologies. As described in further detail below, the data may comprise one or more sub-matrices generated during that application of the SVD to clinical data 12.

Once analysis module 14 has processed clinical data 12 and generated parametric database 16, diagnostic device 6 receives or is otherwise programmed to apply configuration data 13 to assist the diagnosis of patient 8. In the illustrated embodiment, auscultatory sound recording device 18 monitors auscultatory sounds from patient 8, and communicates a digitized representation of the sounds to diagnostic device 6 via communication link 19. Diagnostic device 6 applies configuration data 13 to analyze the auscultatory sounds recorded from patient 8.

In general, diagnostic device 6 applies the configuration data 13 to map the digitized representation received from auscultatory sound recording device 18 to the multi-dimensional energy space computed by data analysis system 4 from clinical data 12. As illustrated in further detail below, diagnostic device 6 applies configuration data 13 to produce a set of vectors within the multidimensional space representative of the captured sounds. Diagnostic device 6 then selects one of the disease regions based on the orientation of the vectors within the multidimensional space relative to the disease regions. In one embodiment, diagnostic device 6 determines which of the disease regions defined within the multidimensional space has a minimum distance from its representative vectors. Based on this determination, diagnostic device presents a suggested diagnosis to clinician 10. Diagnostic device 6 may repeat the analysis for one or more heart cycles identified with the recorded heart sounds of patient 8 to help ensure that an accurate diagnosis is reported to clinician 10.

In various embodiments, diagnostic device 6 may output a variety of message types. For example, diagnostic device 6 may output a “pass/fail” type of message indicating whether the physiological condition of patient 8 is normal or abnormal, e.g., whether or not the patient is experiencing a cardiac pathology. In this embodiment, data analysis system 4 may define the multidimensional space to include two disease regions: (1) normal, and (2) diseased. In other words, data analysis system 4 need not define respective disease regions with the multidimensional space for each cardiac disease. During analysis, diagnostic device 6 need only determine whether the auscultatory sounds of patient 8 more closely maps to the “normal” region or the “diseased” region, and output the pass/fail message based on the determination. Diagnostic device 6 may display a severity indicator based on a calculated distance from which the mapped auscultatory sounds of patient 8 is from the normal region.

As another example, diagnostic device 6 may output diagnostic message to suggest one or more specific pathologies currently being experienced by patient 8. Alternatively, or in addition, diagnostic device 6 may output a diagnostic message as a predictive assessment of a pathology to which patient 8 may be tending. In other words, the predictive assessment indicates whether the patient may be susceptible to a particular cardiac condition. This may allow clinician 8 to proactively prescribe therapies to reduce the potential for the predicted pathology from occurring or worsening.

Diagnostic device 6 may support a user-configurable mode setting by which clinician 10 may select the type of message displayed. For example, diagnostic device 6 may support a first mode in which only a pass/fail type message is displayed, a second mode in which one or more suggested diagnoses is displayed, and a third mode in which one or more predicted diagnoses is suggested.

Diagnostic device 6 may be a laptop computer, a handheld computing device, a personal digital assistant (PDA), an echocardiogram analyzer, or other device. Diagnostic device 6 may include an embedded microprocessor, digital signal processor (DSP), field programmable gate array (FPGA), application specific integrated circuit (ASIC) or other hardware, firmware and/or software for implementing the techniques. In other words, the analysis of auscultatory sounds from patient 8, as described herein, may be implemented in hardware, software, firmware, combinations thereof, or the like. If implemented in software, a computer-readable medium may store instructions, i.e., program code, that can be executed by a processor or DSP to carry out one of more of the techniques described above. For example, the computer-readable medium may comprise magnetic media, optical media, random access memory (RAM), read-only memory (ROM), non-volatile random access memory (NVRAM), electrically erasable programmable read-only memory (EEPROM), flash memory, or other media suitable for storing program code.

Auscultatory sound recording device 18 may be any device capable of generating an electronic signal representative of the auscultatory sounds of patient 8. As one example, auscultatory sound recording device 18 may be an electronic stethoscope having a digital signal processor (DSP) or other internal controller for generating and capturing the electronic recording of the auscultatory sounds. Alternatively, non-stethoscope products may be used, such as disposable/reusable sensors, microphones and other devices for capturing auscultatory sounds.

Application of the techniques described herein allow for the utilization of raw data in unfiltered form. Moreover, the techniques may utilize auscultatory sounds captured by auscultatory sound recording device 18 that is not in the audible range. For example, an electronic stethoscope may capture sounds ranging from 0-2000 Hz.

Although illustrated as separate devices, diagnostic device 6 and auscultatory sound recording device 18 may be integrated within a single device, e.g., within an electronic stethoscope having sufficient computing resources to record and analyze heart sounds from patient 8 in accordance with the techniques described herein. Communication link 19 may be a wired link, e.g., a serial or parallel communication link, a wireless infrared communication link, or a wireless communication link in accordance with a proprietary protocol or any of a variety of wireless standards, such as 802.11 (a/b/g), Bluetooth, and the like.

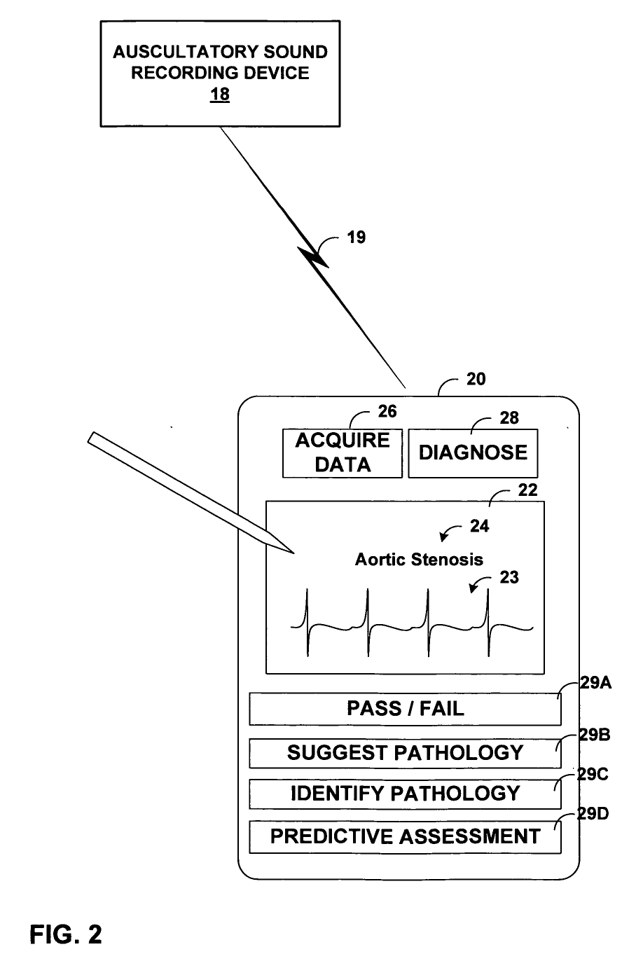

FIG. 2 is a block diagram of an exemplary embodiment of a portable digital assistant (PDA) 20 operating as a diagnostic device to assist diagnosis of patient 8 (FIG. 1). In the illustrated embodiment, PDA 20 includes a touch-sensitive screen 22, input keys 26, 28 and 29A-29D.

Upon selection of acquisition key 26 by clinician 10, diagnostic device 20 enters an acquisition mode to receive via communication link 19 a digitized representation of auscultatory sounds recorded from patient 8. Once the digitized representation is received, clinician 10 actuates diagnose key 28 to direct diagnostic device 20 to apply configuration data 13 and render a suggested diagnosis based on the received auscultatory sounds. Alternatively, diagnostic device 20 may automatically begin processing the sounds without requiring activation of diagnose key 28.

As described in further detail below, diagnostic device 20 applies configuration data 13 to map the digitized representation received from auscultatory sound recording device 18 to the multi-dimensional energy space computed by data analysis system 4. In general, diagnostic device 20 determines to which of the disease regions defined within the multi-dimensional space the auscultatory sounds of patient 8 most closely maps. Based on this determination, diagnostic device 20 updates touch-sensitive screen 22to output one or more suggested diagnoses to clinician 10. In this example, diagnostic device 20 outputs a diagnostic message 24 indicating that the auscultatory sounds indicate that patient 8 may be experiencing aortic stenosis. In addition, diagnostic device may output a graphical representation 23 of the auscultatory sounds recorded from patient 8.

Diagnostic device 20 may include a number of input keys 29A-29D that control the type of analysis performed via the device. For example, based on which of inputs keys 29A-29D has been selected by clinician 10, diagnostic device 20 provides a pass/fail type of diagnostic message, one or more suggested pathologies that patient 8 may currently be experiencing, one or more pathologies that patient 8 has been identified as experiencing, and/or a predictive assessment of one or more pathologies to which patient 8 may be tending.

Screen 22 or an input key could also allow input of specific patient information such as gender, age and BMI (body mass index=weight (kilograms)/height (meters) squared. This information could be used in the analysis set forth here within.

In the embodiment illustrated by FIG. 2, diagnostic device 20 may be any PDA, such as a PalmPilot manufactured by Palm, Inc. of Milpitas, Calif. or a PocketPC executing the Windows CE operating system from Microsoft Corporation of Redmond, Wash.

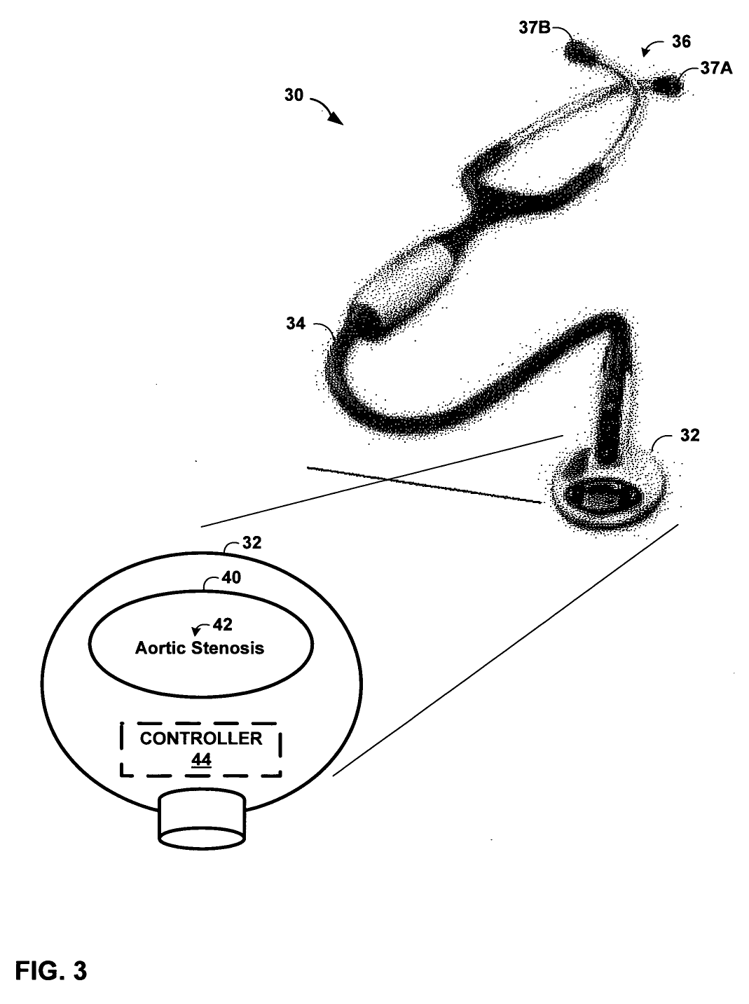

FIG. 3 is a perspective diagram of an exemplary embodiment of an electronic stethoscope 30 operating as a diagnostic device in accordance with the techniques described herein. In the illustrated embodiment, electronic stethoscope 30 comprises a chestpiece 32, a sound transmission mechanism 34 and an earpiece assembly 36. Chestpiece 32 is adapted to be placed near or against the body of patient 8 for gathering the auscultatory sounds. Sound transmission mechanism 34 transmits the gathered sound to earpiece assembly 36. Earpiece assembly 36 includes a pair of earpieces 37A, 37B, where clinician 10 may monitor the auscultatory sounds.

In the illustrated embodiment, chestpiece 32 includes display 40 for output of a diagnostic message 42. More specifically, electronic stethoscope 30 includes an internal controller 44 that applies configuration data 13 to map the auscultatory sounds captured by chestpiece 32 to the multidimensional energy space computed by data analysis system 4. Controller 44 determines to which of the disease regions defined within the energy space the auscultatory sounds of patient 8 most closely maps. Based on this determination, controller 44 updates display 40 to output diagnostic message 42.

Controller 44 is illustrated for exemplary purposes as located within chestpiece 32, and may be located within other areas of electronic stethoscope 30. Controller 44 may comprise an embedded microprocessor, DSP, FPGA, ASIC, or similar hardware, firmware and/or software for implementing the techniques. Controller 44 may include a computer-readable medium to store computer readable instructions, i.e., program code, that can be executed to carry out one of more of the techniques described herein.

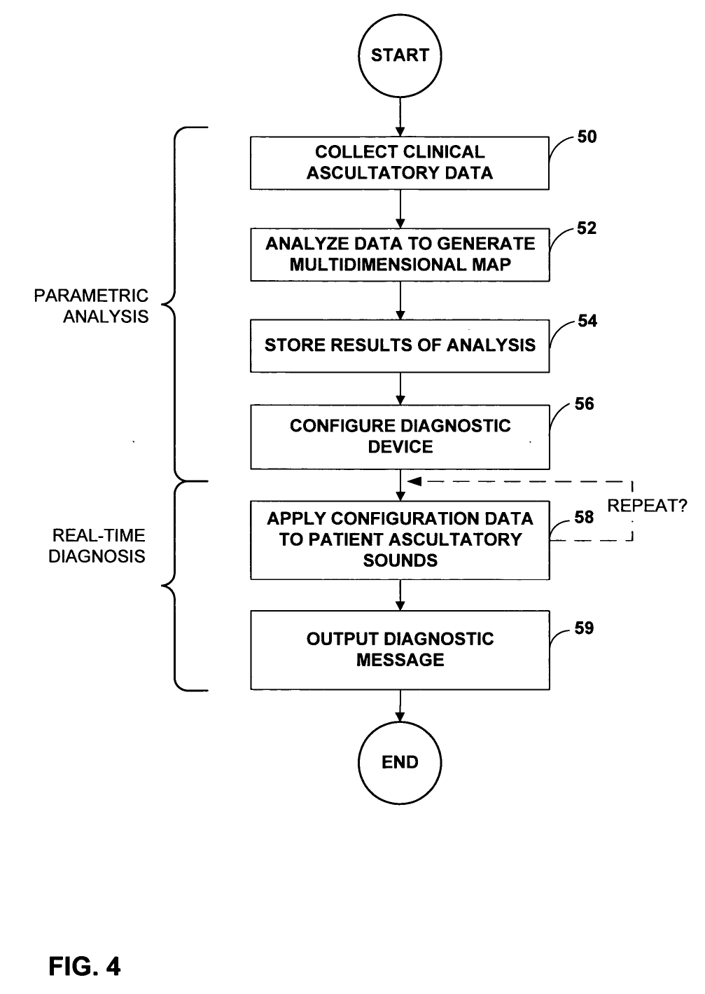

FIG. 4 is a flowchart that provides an overview of the techniques described herein. As illustrated in FIG. 4, the process may generally be divided into two stages. The first stage is referred to as the parametric analysis stage in which clinical data 12 (FIG. 1) is analyzed using SVD to produce configuration data 13 for diagnostic device 6. This process may be computationally intensive. The second stage is referred to as the diagnosis stage in which diagnostic device 6 applies the results of the analysis stage to aid the diagnosis of a patient. For purposes of illustration, the flowchart of FIG. 4 is described in reference to FIG. 1.

Initially, clinical data 12 is collected (50) and provided to data analysis system 4 for singular value decomposition (52). As described above, clinical data 12 comprises electronic recordings of auscultatory sounds from a set of patients having known cardiac conditions.

Analysis module 14 of data analysis system 4 analyzes the recorded heart sounds of clinical data 12 in accordance with the techniques described herein to define a set of disease regions within a multi-dimensional space representative of the electronically recorded heart sounds (52). Each disease region within the multi-dimensional space corresponds to sounds within a heart cycle that have been mathematically identified as indicative of the respective disease. Analysis module 14 stores the results of the analysis within parametric database 16 (54). In particular, the results include configuration data 13 for use by diagnostic device 6 to map patient auscultatory sounds to the generated multidimensional space. Once analysis module 14 has processed clinical data 12, diagnostic device 6 receives or is otherwise programmed to apply configuration data 13 to assist the diagnosis of patient 18 (56). In this manner, data analysis system can be viewed as applying the techniques described herein, including SVD, to analyze a representative sample set of auscultatory sounds recorded from patients having known physiological conditions to generate parametric data that may be applied in real-time or pseudo real-time.

The diagnosis stage commences when auscultatory sound recording device 18 captures auscultatory sounds from patient 8. Diagnosis device 6 applies configuration data 13 to map the heart sounds received from auscultatory sound recording device 18 to the multi-dimensional energy space computed by data analysis system 4 from clinical data 12 (58). For cardiac auscultatory sounds, diagnostic device 6 may repeat the real-time diagnosis for one or more heart cycles identified with the recorded heart sounds of patient 8 to help ensure that an accurate diagnosis is reported to clinician 10. Diagnostic device 6 outputs a diagnostic message based on the application of the configuration and the mapping of the patient auscultatory sounds to the multidimensional space (59).

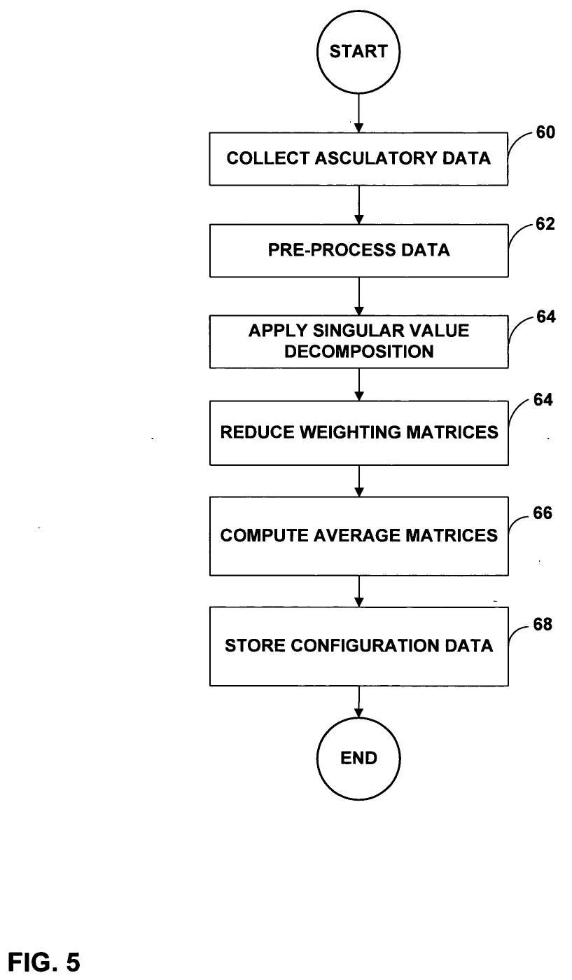

FIG. 5 is a flowchart illustrating the parametric analysis stage (FIG. 4) in further detail. Initially, clinical data 12 is collected from a set of patients having known cardiac conditions (60). In one embodiment, each recording captures approximately eight seconds of auscultatory heart sounds, which represents approximately 9.33 heart cycles for a seventy beat per minute heart rate. Each recording is stored in digital form as a vector R having 32,000 discrete values, which represents a sampling rate of approximately 4000 Hz.

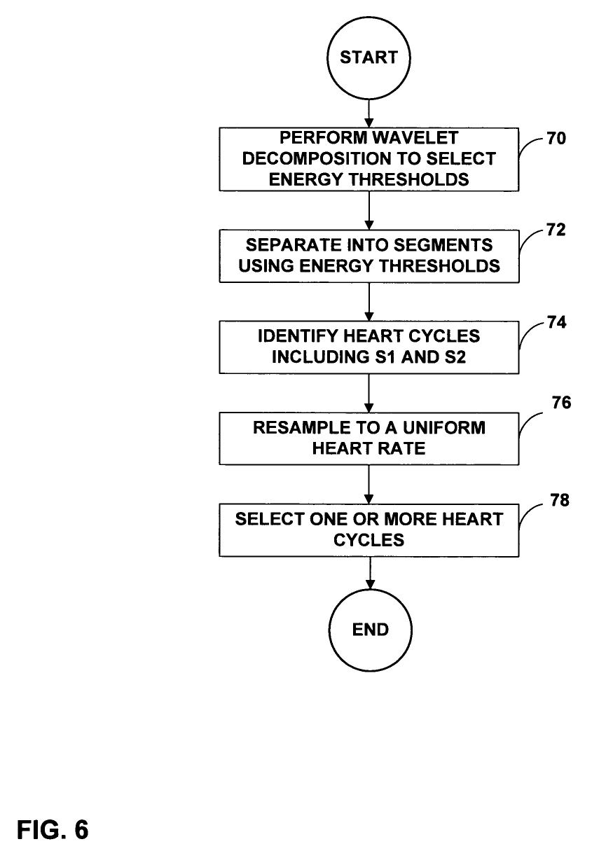

Each heart sound recording R is pre-processed (62), as described in detail below with reference to FIG. 6. During this pre-processing, analysis module 12 processes the vector R to identify a starting time and ending time for each heart cycle. In addition, analysis module 14 identifies starting and ending times for the systole and diastole periods as well as the S1 and S2 periods within each of the heart cycles. Based on these identifications, analysis module 14 normalizes each heart cycle to a common heart rate, e.g., 70 beats per minute. In other words, analysis module 14 may resample the digitized data corresponding to each heart cycle as necessary in order to stretch or compress the data associated with the heart cycle to a defined time period, such as approximately 857 ms, which corresponds to a heart rate of 70 beats per minute.

After pre-processing each individual heart recording, analysis module 14 applies singular value decomposition (SVD) to clinical data 12 to generate a multidimensional energy space and define disease regions within the multi-dimensional energy space that correlate to characteristics of the auscultatory sound (64).

More specifically, analysis module 14 combines N pre-processed sound recordings R for patients having the same known cardiac condition to form an M×N matrix A as follows:

$$A = [ \begin{matrix}1 … & M \\ N… & M\end{matrix} ] ,$$

where each row represents a different sound recording R having M digitized values, e.g., 3400 values.

Next, analysis module 14 applies SVD to decompose A into the product of three sub-matrices:
A=UDV^{T},
where U is an N×M matrix with orthogonal columns, D is an M×M non-negative diagonal matrix and V is an M×M orthogonal matrix. This relationship may also be expressed as:
U^{T}AV=diag(S)=diag(σ₁, . . . , σₚ),
where the elements of matrix S (σ₁, . . . , σₚ) are the singular values of A. In this SVD representation, U is the left singular matrix and V is the right singular matrix. Moreover, U can be viewed as an M×M weighting matrix that defines characteristics with each R that best define the matrix A. More specifically, according to SVD principles, the U matrix provides a weighting matrix that maps the matrix A to a defined region within an M dimensional space.

Analysis module 14 repeats this process for each cardiac condition. In other words, analysis module 14 utilizes sound recordings R for “normal” patients to compute a corresponding matrix A_{NORMAL} and applies SVD to generate a U_{NORMAL} matrix. Similarly, analysis module computes an A matrix and a corresponding U matrix for each pathology. For example, analysis module 14 may generate a U_{AS}, U_{AR}, a U_{TR}, and/or a U_{DISEASED}., where the subscript “AS” designates a U matrix generated from patient or population of patients known by other diagnostic tools to display aortic stenosis. The subscript “AR” designates aortic regurgitation and the subscript “TR” designated tricuspid regurgitation in analogous manner.

Next, analysis module 14 pair-wise multiplies each of the computed U matrices with the other U matrices, and performs SVD on the resultant matrices in order to identify which portions of the U matrices best characterize the characteristics that distinguish between the cardiac conditions. For example, assuming matrices of U_{NORMAL}, U_{AS}, and U_{AR}, analysis module computes the following matrices:
T1=U_{NORMAL}*U_{AS},
T2=U_{NORMAL}*U_{AR}, and
T3=U_{AS}*U_{AR}.

Analysis module 14 next applies SVD on each of the resultant matrices T1, T2 and T3, which again returns a set of sub-matrices that can be used to identify the portions of each original U matrix that maximizes an energy differences within the multidimensional space between the respective cardiac conditions. For example, the matrices computed via applying SVD to T1 can be used to identify those portions of U_{NORMAL} and U_{AS} that maximize the orthogonality of the respective disease regions within the multidimensional space.

Consequently, T1 may be used to trim or otherwise reduce U_{NORMAL} and U_{AS} to sub-matrices that may be more efficiently applied during the diagnosis (64). For example, S matrices computed by application of SVD to each of T1, T2 and T3 may be used. An inverse cosine may be applied to each S matrix to compute an energy angle between the respective two cardiac conditions within the multidimensional space. This energy angle may then be used to identify which portions of each of the U matrices best account for the energy differences between the diseases reasons within the multidimensional space.

Next, analysis module computes an average vector A V for each of the cardiac conditions (66). In particular, for each M×N A matrix formulated from cardiac data 12, analysis module 14 computes a M×N average vector AV that stores the average digitized values computed from the N sound recordings R within the matrix A. For example, analysis module 14 may compute AV_{AS}, AV_{AR}, AV_{TR}, and/or AV_{DISEASED} vectors.

Analysis module 14 stores the computed A V average vectors and the U matrices, or the reduced Umatrices, in parametric database 16 for use as configuration data 13. For example, analysis module 14 may store AV_{AS}, AV_{AR}, AV_{TR}, U_{NORMAL}, U_{AS}, and U_{AR}, for use as configuration data 13 by diagnostic device 6 (68).

FIG. 6 is a flowchart that illustrates in further detail one technique for pre-processing of an auscultatory sound recording R. In general, the pre-processing techniques separate the auscultatory sound recording R into heart cycles, and further separate each heart cycle into four parts: a first heart sound, a systole portion, a second heart sound, and a diastole portion. The pre-processing techniques apply Shannon Energy Envelogram (SEE) for noise suppression. The SEE is then thresholded making use of the relative consistency of the heart sound peaks. The threshold used can be adaptively generated based upon the specific auscultatory sound recording R.

Initially, analysis module 14 performs wavelet analysis on the auscultatory sound recording R to identify energy thresholds within the recording (70). For example, wavelet analysis may reveal energy thresholds between certain frequency ranges. In other words, certain frequency ranges may be identified that contain substantial portions of the energy of the digitized recording.

Based on the identified energy thresholds, analysis module 14 decomposes the auscultatory sound recording R into one or more frequency bands (72). Analysis module 14 analyzes the characteristics of the signal within each frequency band to identify each heart cycle. In particular, analysis module 14 examines the frequency bands to identify the systole and diastole stages of the heart cycle, and the S1 and S2 periods during with certain valvular activity occurs (74). To segment each heart cycle, analysis module 14 may first apply a low-pass filter, e.g., an eight order Chebyshev-type low-pass filter with a cutoff frequency of 1 kHz. The average SEE may then be calculated for every 0.02 second segment throughout the auscultatory sound recording R with 0.01 second segment overlap as follows:

$${E}_{S} = - \frac{1}{N} {\sum }_{i = 1}^{N} {x}_{norm}^{2} ( i ) log {X}_{norm}^{2} ( i )$$

where Xₙₒᵣₘ is the low-pass filtered and normalized sample of the sound recording and N is the number of signal samples in the 0.02 second segment, e.g., N equals 200. The normalized average Shannon Energy versus the time axis may then be computed as:

$${P}_{S} ( t ) = \frac{{E}_{S} ( t ) - M ( {E}_{S} ( t ) )}{S ( {E}_{S} ( t ) )}$$

where M(E_{S}(t)) is the mean of Es(t) and S(E_{S}(t)) is the standard deviation of E_{S}(t). The mean and standard deviation are then used as a basis for identifying the peaks with each heart cycle and the starting and times for each segment with each heart cycle.

Once the starting and ending times for each heart cycle and each S1 and S2 periods is determined within the auscultatory sound recording R, analysis module 14 re-samples the auscultatory sound recording R as necessary to stretch or compress so that each heart cycle and each S1 and S2 period occur over a time period (76). For example, analysis module 14 may normalize each heart cycle to a common heart rate, e.g., 70 beats per minute and may ensure that each S1 and S2 periods within the cycle correspond to an equal length in time. This may advantageously allow the portions of the auscultatory sound recording R for the various phases of the cardiac activity to be more easily and accurately analyzed and compared with similar portions of the other auscultatory sound recordings.

Upon normalizing the heart cycles within the digitized sound recording R, analysis module 14 selects one or more of the heart cycles for analysis (78). For example, analysis module 14 may identify a “cleanest” one of the heart cycles based on the amount of noise present within the heart cycles. As other examples, analysis module 14 may compute an average of all of the heart cycles or an average to two or more randomly selected heart cycles for analysis.

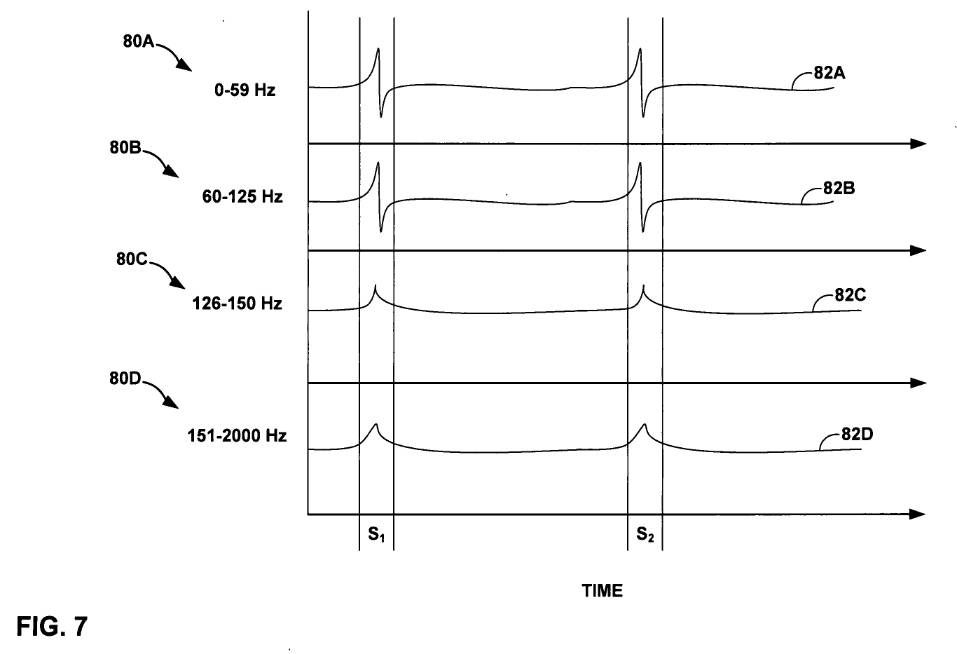

FIG. 7 is a graph that illustrates an example result of the wavelet analysis and energy thresholding described above in reference to FIG. 6. In particular, FIG. 7 illustrates a portion of a sound recording R. In this example, analysis module 14 has decomposes an exemplary auscultatory sound recording R into four frequency bands 80A-80D, and each frequency band includes a respective frequency component 82A-82D.

Based on the decomposition, analysis module 14 detects changes to the auscultatory sounds indicative of the stages of the heart cycle. By analyzing the decomposed frequencies and identifying the relevant characteristics, e.g., changes of slope within one or more of the frequency bands 80, analysis module 14 is able to reliably detect the systole and diastole periods and, in particular, the start and end to the S1 and S2 periods.

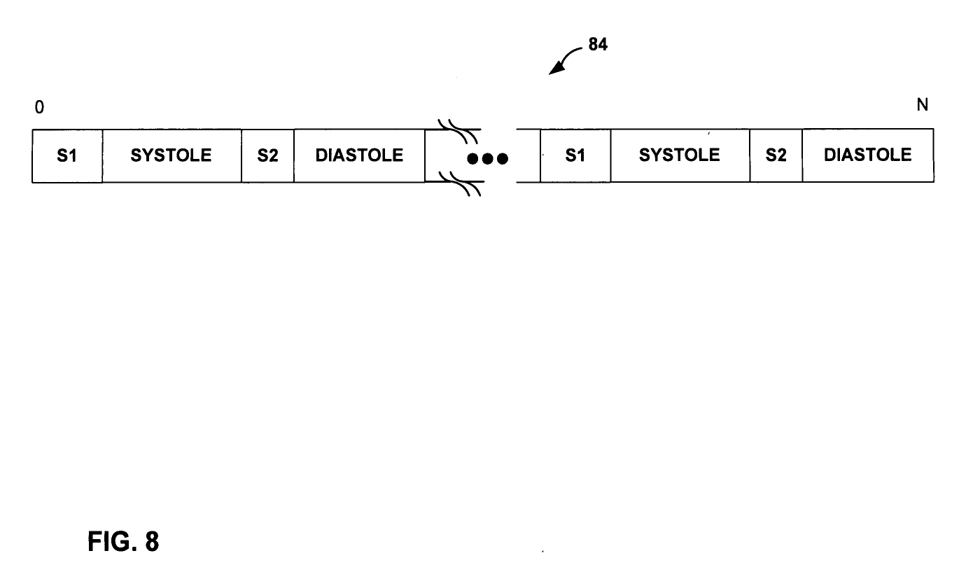

FIG. 8 illustrates an example data structure 84 of an auscultatory sound recording R. As illustrated, data structure 84 may comprise a 1×N vector storing digitized data representative of the auscultatory sound recording R. Moreover, based on the pre-processing and re-sampling, data structure 84 stores data over a fixed number of heart cycles, and each S1 and S2 regions occupy a pre-defined portion of the data structure. For example, S1 region 86 for the first heart cycle may comprise elements 0-399 of data structure 84, and systole region 87 of the first heart cycle may comprises elements 400-1299. This allows multiple auscultatory sound recordings R to be readily combined to form an M×N matrix A, as described above, in which the S1 and S2 regions for a given heart cycle are column-aligned.

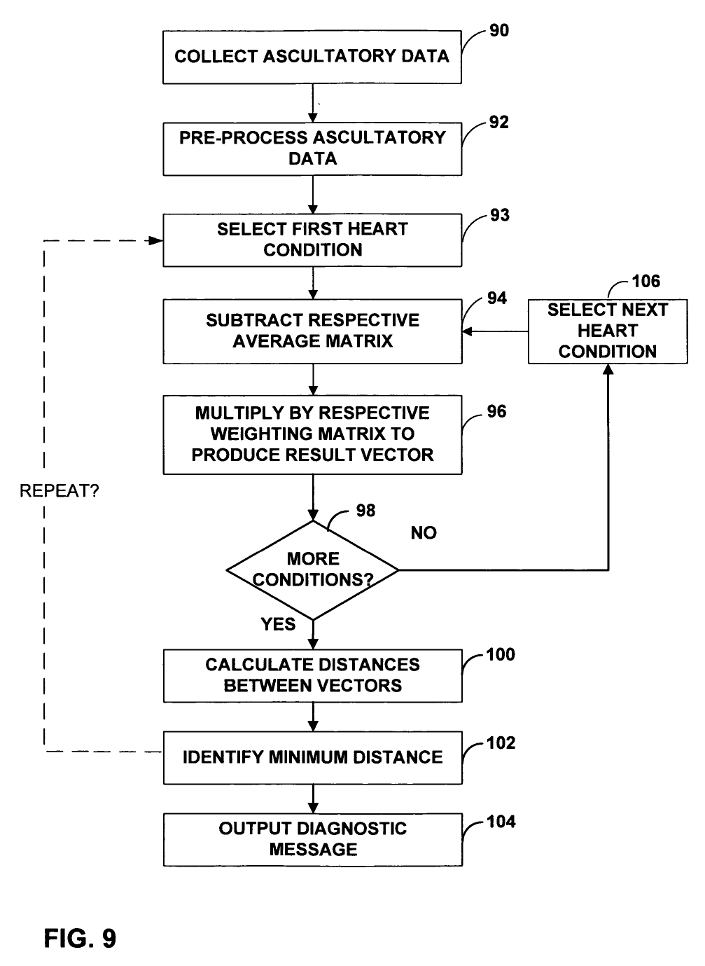

FIG. 9 is a flowchart illustrating the diagnostic stage (FIG. 4) in further detail. Initially, auscultatory data is collected from patient 8 (90). As described above, the auscultatory data may be collected by a separate auscultatory sound recording device 18, e.g., an electronic stethoscope, and communicated to diagnostic device 6 via link communication 19. In another embodiment, the functionality of diagnostic device 6 may be integrated within auscultatory sound recording device 18. Similar to the parametric analysis stage, the collected auscultatory recording captures approximately eight seconds of auscultatory sounds from patient 8, and may be stored in digital form as a vector R_{PAT} having 3400 discrete values.

Upon capturing the auscultatory data R_{PAT}, diagnostic device 6 pre-processes the heart sound recording R_{PAT} (92), as described in detail above with reference to FIG. 6. During this pre-processing, diagnostic device 6 processes the vector R_{PAT} to identify a starting time and an ending time for each heart cycle, and starting and ending times for the systole and diastole periods as well as the S1 and S2 periods of each of the heart cycles. Based on these identifications, diagnostic device 6 normalizes each heart cycle to a common heart rate, e.g., 70 beats per minute.

Next, diagnostic device 6 initializes a loop that applies configuration data 13 for each physiological condition examined during the analysis stage. For example, diagnostic device may utilize configuration data of AV_{AS}, AV_{AR}, AV_{TR}, U_{NORMAL}, U_{AS}, and U_{AR}, to assist diagnosis of patient 8.

Initially, diagnostic device 6 selects a first physiological condition, e.g., normal (93). Diagnostic device 6 then subtracts the corresponding average vector AV from the captured auscultatory sound vector R_{PAT} to generate a difference vector D (94). D is referred to generally as a difference vector as the resulting digitized data of D represents differences between the captured heart sound vector R_{PAT} and the currently selected physiological condition. For example, diagnostic device 6 may calculate D_{NORMAL} as follows:
D_{NORMAL}=R_{PAT}−AV_{NORMAL}.

Diagnostic device 6 then multiples the resulting difference vector D by the corresponding U matrix for the currently selected physiological condition to produce a vector P representative of patient 8 with respect to the currently selected cardiac condition (96). For example, diagnostic device 6 may calculate P_{NORMAL} vector as follows:
P_{NORMAL}=D_{NORMAL}* U_{NORMAL}.
Multiplying the difference vector D via the corresponding U matrix effectively applies a weighting matrix associated with the corresponding disease region within the multi-dimensional space, and produces a vector P within the multidimensional space. The alignment of the vector P relative to the disease region of the current cardiac condition depends on the normality of the resulting difference vector D and the U matrix determined during the analysis stage.

Diagnostic device 6 repeats this process for each cardiac condition defined within the multidimensional space to produce a set of vectors representative of the auscultatory sound recorded from patient 8 (98, 106). For example, assuming configuration data 13 comprises AV_{AS}, AV_{AR}, AV_{TR}, U_{NORMAL}, U_{AS}, and U_{AR}, diagnostic device 6 calculates four patient vectors as follows:
P_{NORMAL}=D_{NORMAL}U_{NORMAL},
P_{AS}=D_{AS}*U_{AS},
P_{AR}=D_{AR}*U_{AR}, and
P_{TR}=D_{TR}*U_{TR}.

This set of vectors represents the auscultatory sounds recorded from patient 8 within the multidimensional space generated during the analysis stage. Consequently, the distance between each vector and the corresponding disease region represents a measure of similarity between the characteristics of the auscultatory sounds from patient 8 and the characteristics of auscultatory sounds of patients known to have the respective cardiac conditions.

Diagnostic device 6 then selects one of the disease regions as a function of the orientation of the vectors and the disease regions within the multidimensional space. In one embodiment, diagnostic device determines which of the disease regions defined within the energy space has a minimum distance from the representative vectors. For example, diagnostic device 6 first calculates energy angles representative of the minimum angular distances between each of the vectors P and the defined disease regions (100). Continuing with the above example, diagnostic device 6 may compute the following four distance measurements:
DIST_{NORMAL}=P_{NORMAL}−MIN[P_{AS}, P_{AR}, P_{TR}],
DIST_{AS}=P_{AS}−MIN [P_{NORMAL}, P_{AR}, P_{TR}],
DIST_{AR}=P_{AR}−MIN [P_{AS}, P_{NORMAL}, P_{TR}], and
DIST_{TR}=P_{TR}−MIN [P_{AS}, P_{AR}, P_{NORMAL}].

In particular, each distance measurement DIST is a two-dimensional distance between the respective patient vector P and the mean of each of the defined disease regions within the multidimensional space.

Based distances, diagnostic device 6 identifies the smallest distance measurement (102) and determines a suggested diagnosis for patient 8 to assist clinician 10. For example, if of the set of patient vectors PAS is the minimum distance away from its respective disease space, the AS disease space, diagnostic device 6 determines that patient 8 may likely be experiencing aortic stenosis. Diagnostic device 6 outputs a representive diagnostic message to clinician 10 based on the identification (104). Prior to outputting the message, diagnostic device 6 may repeat the analysis for one or more heart cycles identified with the recorded heart sounds of patient 8 to help ensure that an accurate diagnosis is reported to clinician 10.

### EXAMPLES

The techniques described herein were applied to clinical data for a set of patients known to have either “normal” cardiac activity or aortic stenosis. In particular, a multidemensional space was generated based on the example clinical data, and then the patients were assessed in real-time according to the techniques described herein.

The following table shows distance calculations for the auscultatory sounds for the patients known to have a normal cardiac conditions. In particular, vectors were computed for each of the measured heart cycles for each patient. Table 1 shows distances for the vectors, measured in volts, with respect to a disease region within the multidimensional space associated with the normal cardiac condition.

TABLE 1

| HEART | PATIENT | PATIENT | PATIENT |
|---|---|---|---|
| CYCLE | 1 | 2 | 3 |
| 1 | 0.45 | 0.25 | 0.20 |
| 2 | 0.64 | 0.14 | 0.18 |
| 3 | 0.38 | 0.21 | 0.32 |
| 4 | | 0.36 | |
| 5 | | 0.20 | |
| 6 | | 0.33 | |

Table 2 shows distance calculations, measured in volts, for the auscultatory sounds for the patients known to have aortic stenosis. In particular, Table 2 shows energy distances for the vectors with respect to a region within the multidimensional space associated with the aortic stenosis cardiac condition.

TABLE 2

| HEART CYCLE | PATIENT 4 | PATIENT 5 |
|---|---|---|
| 1 | −0.43 | −0.49 |
| 2 | −0.67 | −0.43 |
| 3 | −0.55 | −0.37 |
| 4 | −0.43 | −0.64 |
| 5 | −0.34 | −0.17 |
| 6 | −0.44 | −0.14 |
| | −0.60 | |

As illustrated by Table 1 and Table 2, the vectors are clearly separate within the multidimensional space, an indication that diagnosis can readily be made. All five patients followed a similar pattern.

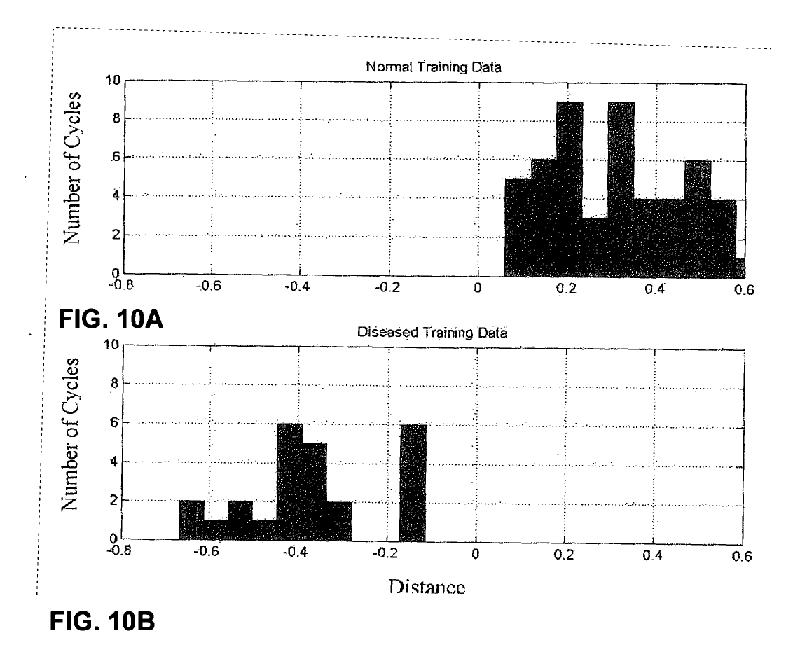

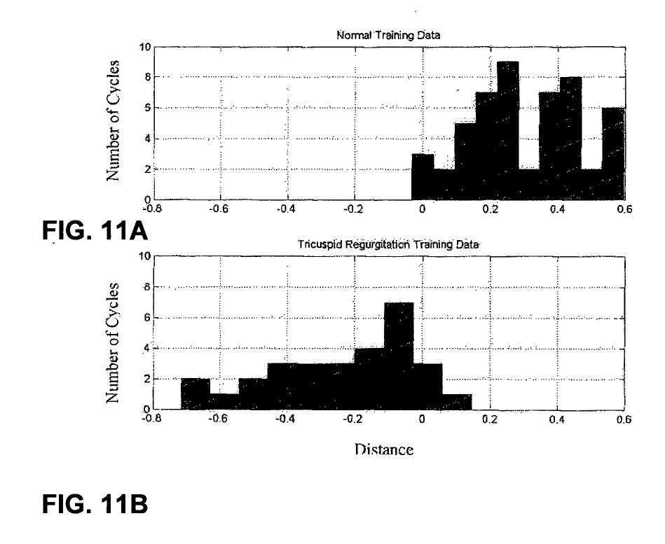

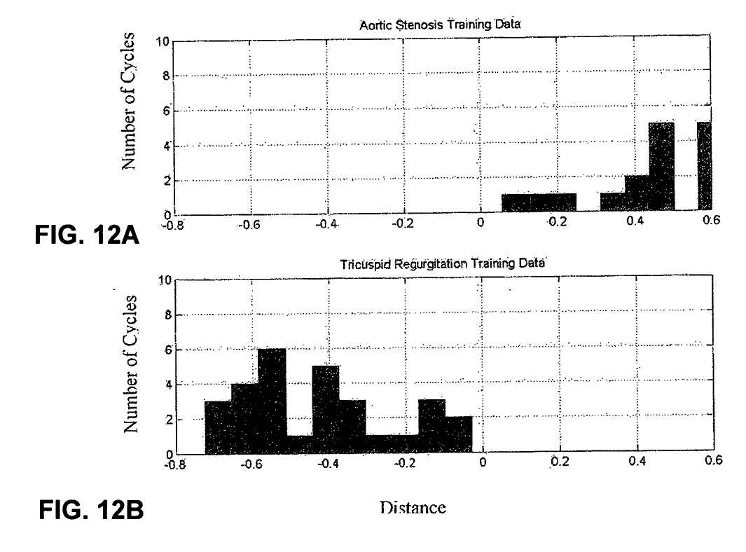

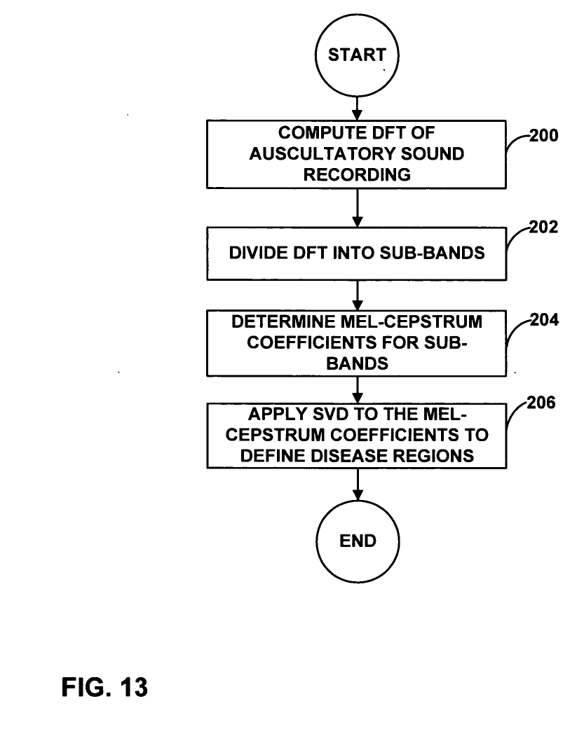

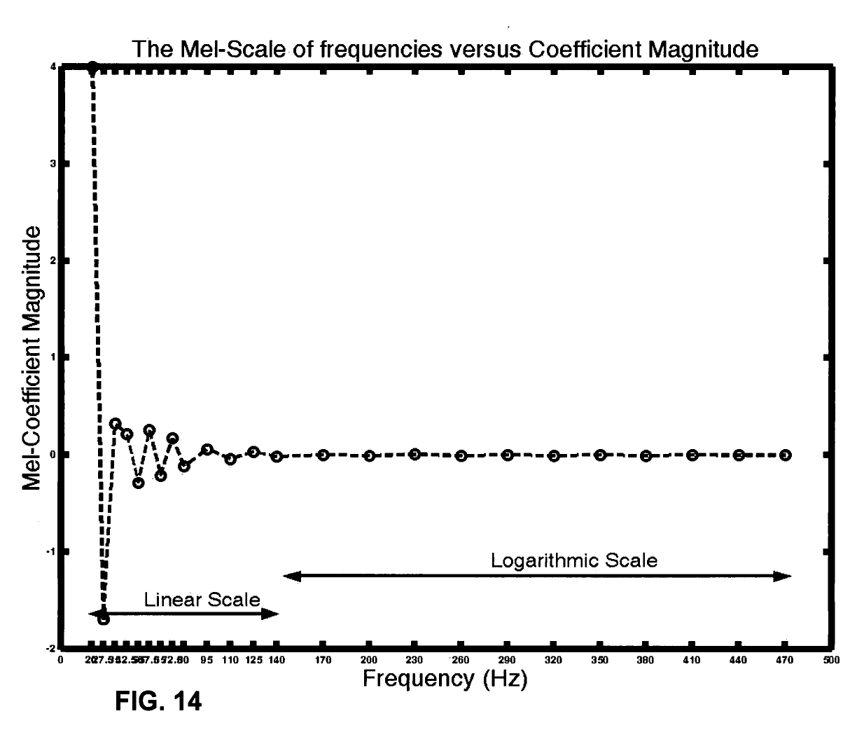

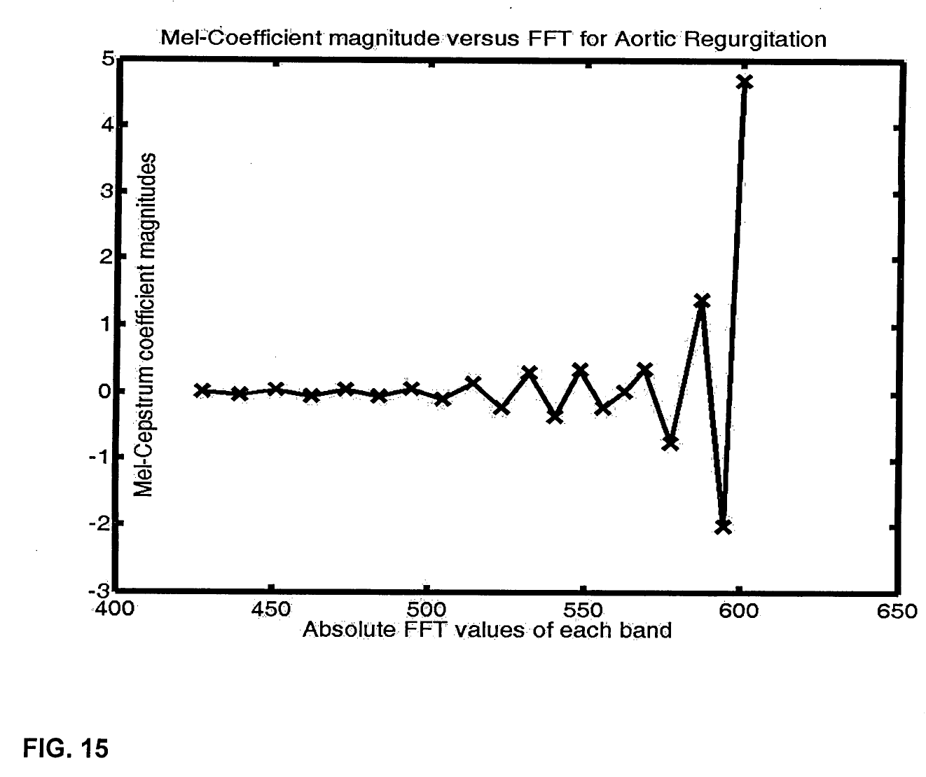

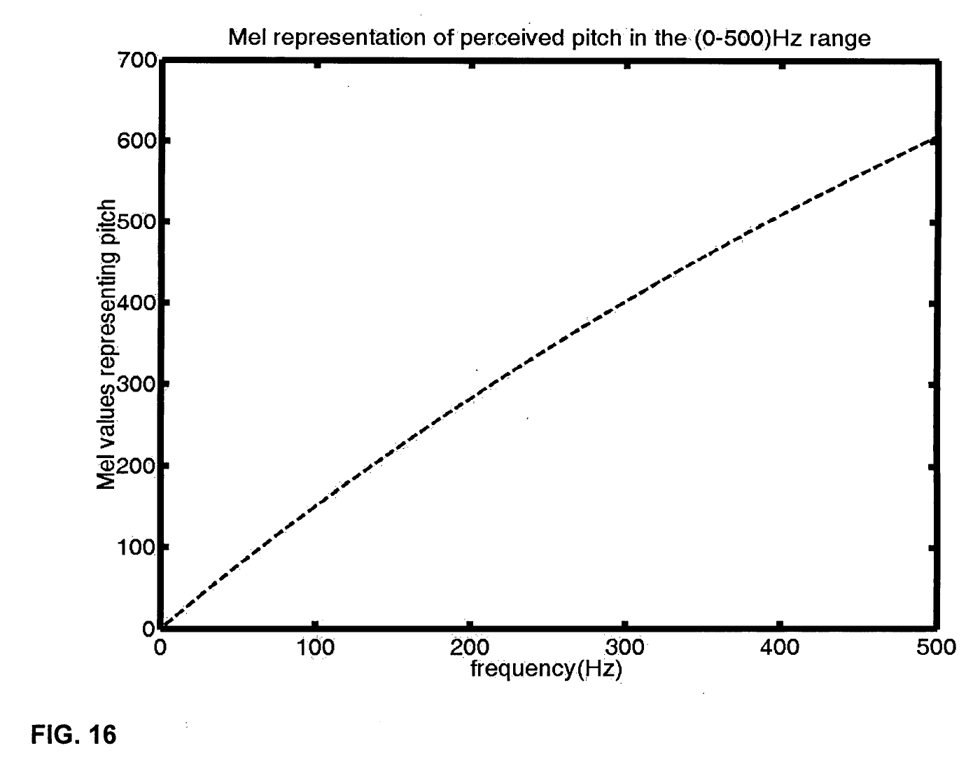

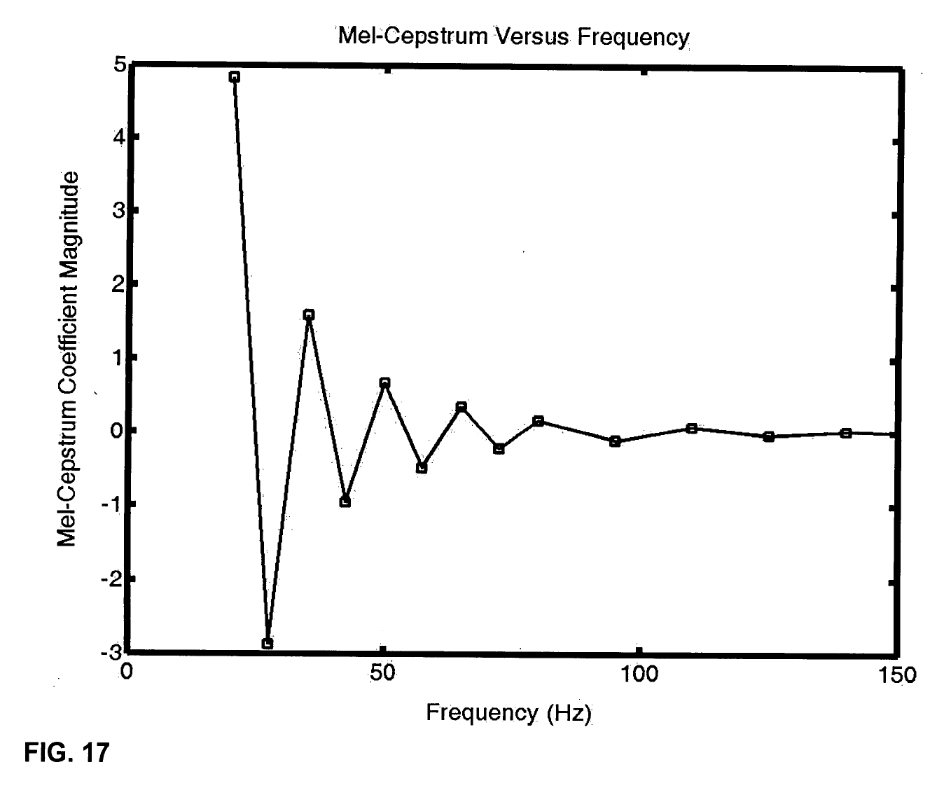

FIGS 10A and 10B are graphs that generally illustrate the exemplary results. In particular, FIGS 10A and 10B illustrate aortic stenosis data compared to normal data. Similary, FIGS. 11A and 11B are graphs that illustrate tricuspid regurgitation data compared to normal data, FIGS 12A and 12B are graphs that illustrate aortic stenosis data compared to tricuspid regurgitation data. In general, the graphs of FIGS. 10A, 10B, 11A, and 11B illustrate that the techniques result in substantially non-overlapping data for the normal data and disease-related data.

FIG. 13 is a flowchart that illustrates another technique for pre-processing of an auscultatory sound recording R. In particular, FIG. 14 describes application of voice recognition techniques to generate mel-cepstrum coefficients for use by the SVD process described herein or other principle component analysis technique. Unlike the pre-processing technique described with respect to FIG. 6, application of voice recognition technology to the auscultatory sound recording R may eliminate the need to separate the auscultatory sound recording R into heart cycles, and further separate each heart cycle into four parts: a first heart sound, a systole portion, a second heart sound, and a diastole portion. Segmentation may be computationally intensive and time-consuming.

In general, a cepstrum is a discrete cosine transform of a log-spectrum of a signal and is commonly used in speech recognition systems. A mel-cepstrum is a modified version of the cepstrum and was designed to exploit the human auditory system by dividing the frequency domain in a non-uniform manner during cepstrum computation.

First, analysis module 14 computes a Discrete Fourier transform (DFT) of auscultatory sound recording R using an FFT algorithm and a hanning window (200). Next, analysis module 14 divides the DFT(R) into M non-uniform sub-bands throughout the audible range (202). In particular, analysis module 14 may split lower frequency portion of the audible range into N equal sub-bands. For example, may split the frequency range of 20-500 Hz linearly into 12 sub-bands. Next, split the upper frequency band logarithmically into N sub-bands. For example, may split 500 to 200 Hz logarithmically into 12 sub-bands. One reason for such a split is because audible components within the higher frequency band may be noise.

Analysis module 14 then formulates the resultant signal as a magnitude-frequency representation and determines mel-cepstrum coefficients for each of the defined sub-bands (204). A mel-cepstrum vector (c=[c1, c2, . . . , cK] can be computed from the discrete cosine transform (DCT) of the auscultatory sound vecrtor R as follows:

$${C}_{k} = {\sum }_{i = 1}^{M} log ( {e}_{i} ) cos [ \frac{k ( i - 0.5 ) π}{M} ] , k = 1 , 2 , … , K$$

where M represents the number of sub-bands.

In particular, analysis module 14 selects the components of the mel-cepstrum coefficients that are most representative of variability of between the disease states and uses those coefficients as inputs to the SVD process described herein to define the disease regions and their boundaries within the multidimensional space (206). In this case, the SVD analysis utilizes a vector of the determined mel-cepstrum coefficients instead of using an auscultatory sound vector. One example of Mel-cepstrum-based Principle Component Analysis is described in “Classification of Closed- and Open-Shell Pistachio Nuts Using Voice-Recognition Technology,” A. E. Cetin et al., Transactions of ASAE, Vol. 47(2): 659-664, 2004, hereby incorporated by reference. In other embodiments, all parametric and non-parametric techniques, such as the use of regressive modeling, neural networks or expert systems for feature extraction.

FIGS. 14-17 are graphs that illustrate exemplary mel-cepstrum coefficients for a single disease state, aortic regurgitation in this example. In particular, FIG. 14 is a graph that plots magnitudes of the mel-cepstrum coefficients determined over a frequency range of zero to 500 Hz. As illustrated, the techniques utilize a linear scale for the sub-bands for lower frequencies (e.g., 0 to 140 Hz) and a log scale for higher frequencies (e.g., 140-500 Hz).

FIG. 15 is a graph that plots magnitudes of the mel-cepstrum coefficients for aortic regurgitation versus FFT values for each frequency band.

FIG. 16 is a graph that plots perceived pitch for the mel-cepstrum representation over a frequency range of zero to 500 Hz.

FIG. 17 is a graph that plots magnitudes of the mel-cepstrum coefficients determined for an exemplary disease region over a frequency range of zero to 500 Hz.

Various embodiments of the invention have been described. For example, although described in reference to sound recordings, the techniques may be applicable to other electrical recordings from a patient. The techniques may be applied, for example, to electrocardiogram recordings electrically sensed from a patient. These and other embodiments are within the scope of the following claims.

1. A method comprising:
applying voice recognition to auscultatory sounds associated with known physiological conditions to generate voice recognition coefficients; and
mapping the coefficients to a set of one or more disease regions defined within a multidimensional space.

2. The method of claim 1, wherein applying voice recognition comprises:
dividing each of the auscultatory sounds into a plurality of sub-bands; and
computing mel-cepstrum coefficients for the sub-bands.

3. The method of claim 1, further comprising outputting a diagnostic message associated with a physiological condition of the patient as a function of the coefficients and the disease regions defined within the multidimensional space.

4. The method of claim 3, wherein outputting a diagnostic message comprises:
selecting one of the disease regions of the multidimensional space; and
outputting the diagnostic message based on the selection.

5. The method of claim 4, wherein selecting one of the disease regions comprises:
computing a plurality of vectors within the multidimensional space from the vectors of coefficients;
identifying which of the vectors has a minimum distance from its respective disease region; and
selecting the disease region associate with the identified vectors.

6. The method of claim 1, wherein each disease region within the multi-dimensional space is defined by characteristics of the auscultatory sounds associated with the known physiological conditions that have been identified as indicators for the respective physiological condition.

7. The method of claim 3, wherein outputting a diagnostic message comprises outputting a pass/fail message that indicates whether an abnormal physiological condition has been detected.

8. The method of claim 3, wherein outputting a diagnostic message comprises outputting a diagnostic message identifying one or more specific pathologies currently being experienced by patient.

9. The method of claim 3, wherein outputting a diagnostic message comprises outputting the diagnostic message to indicate the patient is susceptible to one or more of the physiological conditions.

10. The method of claim 3, wherein outputting a diagnostic message comprises selecting a message type for the diagnostic message based on a user configurable mode.

11. The method of claim 3, wherein the message type comprises one of a pass/fail message type, a suggested diagnosis message type, and a predictive diagnosis message type.

12. The method of claim 5, further comprising outputting a severity indicator based on a calculated distance from at least one of the vectors and a normal region within the multidimensional space.

13. The method of claim 1, wherein mapping auscultatory sounds comprises:
formulating a set of matrices that store the coefficients, wherein each matrix is associated with a different one of the physiological conditions; and
applying singular value decomposition (“SVD”) to each of the matrices to compute respective sets of sub-matrices that define the disease regions within the multidimensional space.

14. The method of claim 13, wherein formulating a set of matrices comprises formulating the set of matrices to store digitized representations in a raw format that has not been filtered.

15. The method of claim 13, further comprising storing at least a portion of one or more of the sub-matrices within a database for use as configuration data for a diagnostic device.

16. The method of claim 13, further comprising:
programming a diagnostic device in accordance with configuration data generated by the application of SVD to the set of matrices, wherein the configuration data includes at least one of the sub-matrices associated with the different physiological conditions; and
applying the configuration data with the diagnostic device to a digitized representation of the auscultatory sounds associated with the patient to produce the vectors within the multidimensional space.

17. The method of claim 13, wherein applying SVD comprises applying SVD to decompose a matrix A of the set of matrices into the product of three sub-matrices as:
A=UDV^{T},
where U is an N×M matrix with orthogonal columns, D is an M×M non-negative diagonal matrix and V is an M×M orthogonal matrix.

18. The method of claim 17, further comprising:
computing a set of matrices T by pair-wise multiplying each of the computed U matrices with the other U matrices;
performing SVD on each of the resultant matrices T to decompose each matrix T into a respective set of sub-matrices; and
applying the sub-matrices generated from each of the matrices T to identify portions of the U matrices to be used in diagnosis of the patient.

19. The method of claim 18, wherein applying the sub-matrices generated from each of the matrices T comprises applying the sub-matrices generated from each of the matrices T to identify portions of the U matrices that maximize the orthogonality of the respective disease regions within the multidimensional space.

20. The method of claim 13, further comprising computing:
computing respective average vectors from the set of matrices, wherein each average vector represents an average of the digitized representations of the auscultatory sounds associated with the respective physiological conditions; and
applying the average vectors and the configuration data with the diagnostic device to the auscultatory sounds associated with the patient to generate the set of vectors within the multidimensional space.

21. The method of claim 20, wherein applying the average vectors and the configuration data with the diagnostic device comprises:
subtracting the corresponding average vectors from a vector representing the auscultatory sounds associated with the patient to generate a set of difference vectors, wherein each difference vector corresponds to a different one of the disease regions in the multi-dimensional space; and
applying the sub-matrices of the configuration data to the difference vectors to generate the vectors representative of the auscultatory sounds associated with the patient.

22. The method of claim 21, wherein applying the sub-matrices of the configuration data comprises multiplying the difference vectors by the corresponding one of the U sub-matrices to produce a respective one of the vectors representative of the auscultatory sounds associated with the patient.

23. The method of claim 1, wherein mapping auscultatory sounds comprises applying principle component analysis to the voice recognition coefficients to define the disease regions and their boundaries within the multidimensional space.

24. The method of claim 1, wherein mapping auscultatory sounds comprises applying the voice recognition coefficients to a neural network to define the disease regions and their boundaries within the multidimensional space.

25. The method of claim 1, wherein each of the auscultatory sounds associated with known physiological conditions comprises a digitized representation of sounds recorded over a plurality of heart cycles.

26. The method of claim 1, wherein the physiological conditions include one or more of a normal physiological condition, aortic regurgitation, aortic stenosis, tricuspid regurgitation, tricuspid stenosis, pulmonary stenosis, pulmonary regurgitation, mitrial regurgitation, aortic aneurisms, carotid artery stenosis and mitrial stenosis.

27. The method of claim 1, further comprising:
capturing the auscultatory sounds associated with the patient using a first device;
communicating a digitized representation of the captured auscultatory sounds from the first device to a second device;
analyzing the digitized representation with the second device to generate the coefficients; and
outputting the diagnostic message with the second device.

28. The method of claim 27, wherein the first device comprises an electronic stethoscope.

29. The method of claim 27, wherein the second device comprises one of a mobile computing device, a personal digital assistant, and an echocardiogram analyzer.

30. The method of claim 1, further comprising:
capturing the auscultatory sounds associated with the patient using an electronic stethoscope;
analyzing the digitized representation with the electronic stethoscope to generate the coefficients; and
outputting the diagnostic message to a display of the electronic stethoscope.

31. The method of claim 1, wherein the physiological conditions comprise cardiac conditions and the auscultatory sounds associated with the patient comprises heart sounds.

32. The method of claim 1, wherein the auscultatory sounds associated with the patient comprises lungs sounds.

33. A method comprising:
applying voice recognition and singular value decomposition (“SVD”) to digitized representations of auscultatory sounds associated with physiological conditions to map the auscultatory sounds to a set of one or more disease regions within a multidimensional space; and
outputting configuration data for application by a diagnostic device based on the multidimensional mapping.

34. The method of claim 33, wherein applying voice recognition and SVD further comprises:
formulating a set of matrices that store digitized representations of the auscultatory sounds associated with the physiological conditions, wherein each matrix is associated with a different one of the physiological conditions and stores the digitized representations of the auscultatory sounds associated with the respective physiological condition; and
applying SVD to each of the matrices to decompose the matrices into respective sets of sub-matrices that define the disease regions within the multidimensional space.

35. The method of claim 34, wherein outputting configuration data comprises storing at least a portion of one or more of the sub-matrices for each of the physiological conditions within a database.

36. The method of claim 34, wherein applying voice recognition and SVD comprises applying SVD to decompose a matrix A of the set of matrices into the product of three sub-matrices as:
A=UDV^{T},
where U is an N×M matrix with orthogonal columns, D is an M×M non-negative diagonal matrix and V is an M×M orthogonal matrix.

37. The method of claim 36, further comprising:
computing a set of matrices T by pair-wise multiplying each of the computed U matrices with the other U matrices;
performing SVD on each of the resultant matrices T to decompose each matrix T into a respective set of sub-matrices; and
applying the sub-matrices generated from each of the matrices T to identify portions of the U matrices to be used in diagnosis of the patient.

38. The method of claim 37, wherein applying the sub-matrices generated from each of the matrices T comprises applying the sub-matrices generated from each of the matrices T to identify portions of the U matrices that maximize the orthogonality of the respective disease regions within the multidimensional space.

39. The method of claim 34, further comprising computing:
computing respective average vectors from the set of matrices, wherein each average vector represents an average of the digitized representations of the auscultatory sounds associated with the respective physiological conditions; and
generating the configuration data to include the average vectors.

40. The method of claim 33, wherein the physiological conditions include one or more of a normal physiological condition, aortic regurgitation, aortic stenosis, tricuspid regurgitation, tricuspid stenosis, pulmonary stenosis, pulmonary regurgitation, mitrial regurgitation, aortic aneurisms, carotid artery stenosis and mitrial stenosis.

41. The method of claim 33, wherein applying voice recognition and SVD comprise:
dividing each of the auscultatory sounds into a plurality of sub-bands; and
computing mel-cepstrum coefficients for the sub-bands.

42. A method comprising:
storing within a diagnostic device configuration data generated by the application of voice recognition techniques and principle component analysis (PCA) to digitized representations of electrical recordings associated with physiological conditions, wherein the configuration data maps the electrical recordings to a set of one or more disease regions within a multidimensional space;
applying the configuration data to a digitized representation of an electrical recording associated with a patient to select one of the physiological conditions; and
outputting a diagnostic message indicating the selected one of the physiological conditions.

43. The method of claim 42,
wherein applying the configuration data comprises applying the configuration data to the digitized representation representative of the auscultatory sounds associated with the patient to generate a set of one or more vectors within the multidimensional space; and
wherein outputting a diagnostic message comprises outputting a diagnostic message as a function of the vectors and the disease regions defined within the multidimensional space.

44. The method of claim 43,
wherein applying the configuration data comprises selecting one of the disease regions of the multidimensional space as a function of orientations of the vectors within the multidimensional space; and
wherein outputting the diagnostic message comprises outputting the diagnostic message based on the selection.

45. The method of claim 43, wherein each of the vectors correspond to a respective one of the disease regions, and wherein selecting one of the disease regions comprises selecting one of the disease regions as a function of a distance between each of the vectors and the respective disease region.

46. The method of claim 43, wherein the configuration data comprises a sub-matrix generated by the application of PCA to the digitized representations of the auscultatory sounds associated with the known physiological conditions.

47. The method of claim 43, wherein the electrical recordings comprises echocardiograms.

48. The method of claim 43, wherein the electrical recordings comprises digitized representation of auscultatory sounds.

49. A diagnostic device comprising:
a medium that stores data generated by the application of voice recognition to digitized representations of auscultatory sounds associated with known physiological conditions; and
a control unit that applies the configuration data to a digitized representation representative of auscultatory sounds associated with a patient to select one of the physiological conditions, wherein the control unit outputs a diagnostic message indicating the selected one of the physiological conditions.

50. The diagnostic device of claim 49,
wherein the control unit applies the configuration data to the digitized representation representative of the auscultatory sounds associated with the patient to generate a set of one or more vectors within a multidimensional space having a set of defined disease regions, and
wherein the control unit selects one of the physiological conditions based on orientations of the vectors relative to the disease regions within the multidimensional space.

51. The diagnostic device of claim 50, wherein each of the vectors correspond to a respective one of the disease regions, and wherein the control unit selects one of the disease regions as a function of a distance between each of the vectors and the respective disease region.

52. The diagnostic device of claim 50, wherein the configuration data comprises a sub-matrix generated by the application of SVD to the digitized representations of the auscultatory sounds associated with the known physiological conditions.

53. The diagnostic device of claim 49, wherein the diagnostic device comprises one of a mobile computing device, a personal digital assistant, an echocardiogram analyzer, and an electronic stethoscope.

54. A data analysis system comprising:
an analysis module to apply voice recognition and principle component analysis (PCA) to digitized representations of electrical recordings associated with known physiological conditions to map the auscultatory sounds to a set of one or more disease regions within a multidimensional space; and
a database to store data generated by analysis module.

55. The method of claim 54, wherein the electrical recordings comprises echocardiograms.

56. The method of claim 54, wherein the electrical recordings comprises digitized representation of auscultatory sounds.

57. The data analysis system of claim 54,
wherein the analysis module formulates a set of matrices that store the digitized representations of the auscultatory sounds associated with the physiological conditions, wherein each matrix is associated with a different one of the physiological conditions and stores the digitized representations of the auscultatory sounds associated with the respective physiological condition, and
wherein the analysis module applies PCA to each of the matrices to decompose the matrices into respective sets of sub-matrices that define the disease regions within the multidimensional space, and
wherein the analysis module stores within the database at least one of the sub-matrices for each of the disease regions.

58. A computer-readable medium comprising instructions that cause a processor to:
apply configuration data to a digitized representation representative of auscultatory sounds associated with a patient to select one of a set of physiological conditions, wherein the configuration maps the auscultatory sounds to a set of one or more disease regions within a multidimensional space using voice recognition and principle component analysis (PCA); and
output a diagnostic message indicating the selected one of the physiological conditions.

59. The computer-readable medium of claim 58 further comprising instructions to cause the processor to:
apply the configuration data to the digitized representation representative of the auscultatory sounds associated with the patient to generate a set of one or more vectors within the multidimensional space;
select one of the disease regions of the multidimensional space as a function of orientations of the vectors relative to the disease regions within the multidimensional space; and
output the diagnostic message based on the selection.
